(19) United States
(12) Patent Application Publication
(10) Pub. No.: US 2025/0128082 A1
(43) Pub. Date: Apr. 24, 2025

(54) PHOTOTHERAPY CARE TOOTHBRUSH

(71) Applicant: Shenzhen Besman Electronic Technology Co.,LTD, Shenzhen (CN)

(72) Inventor: Xuewu Wang, Shenzhen (CN)

(21) Appl. No.: 19/001,455

(22) Filed: Dec. 25, 2024

(30) Foreign Application Priority Data
Nov. 6, 2024 (CN) 202411572554.9

(51) Int. Cl. A61N 5/06; A46B 13/02; A46B 15/00; A46D 1/00; A61C 17/22

(52) U.S. Cl. CPC A61N 5/0603; A46B 13/02; A46B 15/0055; A46D 1/02; A61C 17/221; A61N 2005/0606; A61N 2005/063; A61N 2005/0644; A61N 2005/0651; A61N 2005/0663; A61N 2005/0666

(57) ABSTRACT

A phototherapy care toothbrush is provided. The phototherapy LEDs are arranged in the toothbrush head body. The phototherapy LEDs are used to emit phototherapy ray with a specific wavelength to assist in oral cleaning and oral care, so that a deep cleaning and care effect can be achieved. Furthermore, redundant chemical medicines can be effectively prevented from entering an internal system of a person from the mouth to harm the health. In addition, in conjunction with various optimization solutions, such as using the bristle base made of a light-transmittance material to refract and guide light to adjust an illumination region, using bristles made of light guide fibers to perform low-loss and high-efficiency light guidance, and covering a light reflection layer at an inner surface of the toothbrush head accommodating chamber to enhance the illumination intensity, the oral phototherapy effect is greatly enhanced.

### TECHNICAL FIELD

The present disclosure relates to the technical field of toothbrushes, and in particular, to a phototherapy care toothbrush.

### BACKGROUND

A toothbrush is a cleaning product that is a handle-style brush. Toothpaste is added onto the toothbrush, and then the toothbrush is used to repeatedly clean all parts of teeth to maintain oral hygiene.

The toothbrush can achieve a good cleaning effect on stains on surfaces of the teeth, but it is not good enough for deeper oral cleaning and care. For example, for teeth penetrated with yellow stains, it is impossible to remove the yellow stains obviously through the toothpaste or essence. For another example, a treatment effect may often be achieved on periodontitis, gingivitis, and the like by taking medicines or spraying anti-inflammatory fluid. Although these traditional methods can eliminate the annoyance of users to an extent, the essence, the medicines, and the anti-inflammatory fluid used in a mouth may more or less enter the internal organs of people through the mouth, thus causing harm to the health. It is not recommended if it is unnecessary.

Therefore, the traditional toothbrushes cannot meet more oral care needs of users in addition to their basic cleaning functions.

### SUMMARY

The present disclosure aims to provide a phototherapy care toothbrush. Phototherapy LEDs are arranged in a toothbrush head body. The phototherapy LEDs are used to emit phototherapy ray with a specific wavelength to assist in oral cleaning and oral care, so that a deep cleaning and care effect can be achieved. Furthermore, redundant chemical medicines can be effectively prevented from entering an internal system of a person from the mouth to harm the health, and the problems mentioned in the background section are solved.

To achieve the above objectives, the present disclosure provides the following technical solution: A phototherapy care toothbrush includes a toothbrush head and a toothbrush handle. The toothbrush head is detachably connected to the toothbrush handle; a toothbrush head body is arranged at one end of the toothbrush head, and a connecting end piece is arranged at the other end; the toothbrush head is detachably connected to an output shaft of a driving motor on the toothbrush handle through the connecting end piece; the toothbrush head body includes a toothbrush head accommodating chamber and a bristle base covered at an opening of the toothbrush head accommodating chamber; bristles are arranged on one side of the bristle base away from the toothbrush head accommodating chamber; a light emission lamp panel is arranged in the toothbrush head accommodating chamber; the light emission lamp panel is encapsulated with phototherapy LEDs; a direction of light emission of the phototherapy LEDs is towards one side of the bristle base; the bristle base is a light-transmittance member, and the bristles are light guide fibers; and the toothbrush head and toothbrush handle are electrically connected to each other through an electrical connection mechanism.

Preferably, the bristle base is sealed at the opening of the toothbrush head accommodating chamber by water.

Preferably, ends of the bristles away from the toothbrush head accommodating chamber extend out and are placed outside the bristle base, and ends facing the toothbrush head accommodating chamber penetrate through the bristle base and are communicated to the toothbrush head accommodating chamber.

Preferably, an inner surface of the toothbrush head accommodating chamber is covered with a light reflection layer.

Preferably, the phototherapy LEDs include blue-light LEDs and red-light LEDs; a light wavelength of the blue-light LEDs is 400 to 460 nm; and a light wavelength of the red-light LED is 630 to 660 nm.

Preferably, the electrical connection mechanism includes a power receiving coil arranged in the toothbrush head and a power transmitting coil arranged on the toothbrush handle; and the power receiving coil and the power transmitting coil are wirelessly electrically connected to each other.

Preferably, the toothbrush head includes a toothbrush head housing; the toothbrush head accommodating chamber is formed in one end of the toothbrush head housing, and a conical cavity is formed in the other end; the connecting end piece is embedded in the conical cavity; the power receiving coil is arranged on one side of the connecting end piece facing the conical cavity; the toothbrush handle includes a handle housing; the handle housing is a sleeve with openings provided in two ends; an upper end plug is arranged at the opening in the upper end of the handle housing; the driving motor is mounted in the handle housing; an output shaft of the driving motor penetrates through the upper end plug and extends outwards from the opening in the upper end of the handle housing; the upper end plug is provided with the power transmitting coil; and the power receiving coil is wirelessly electrically connected to the power transmitting coil.

Preferably, an electrical appliance support is arranged in the handle housing; a circuit board assembly and a storage battery are arranged in the handle housing; the driving motor, the storage battery, and the circuit board assembly are fixedly mounted on the electrical appliance support; a waterproof cover is encapsulated at the opening in the upper end of the handle housing; the waterproof cover sleeves the output shaft of the driving motor in a circumferentially direction and is clamped between the upper end plug and the handle housing; a bottom end plug is encapsulated at the opening in the lower end of the handle housing; a power input port is arranged on the bottom end plug; and the power input port is electrically connected to the circuit board assembly.

Preferably, the circuit board assembly is electrically connected with control buttons and indicator lights; the control buttons and the indicator lights are arranged in preset through holes of the handle housing.

Preferably, the electrical appliance support includes a motor support and a battery support; and an end portion of the motor support and an end portion of the battery support opposite to the end portion of the motor support re connected in an inserted manner.

Compared with the prior art, the present disclosure has the beneficial effects below:

According to the present disclosure, phototherapy LEDs are arranged in a toothbrush head body. The phototherapy LEDs are used to emit phototherapy ray with a specific wavelength to assist in oral cleaning and oral care, so that a deep cleaning and care effect can be achieved. Furthermore, redundant chemical medicines can be effectively prevented from entering an internal system of a person from the mouth to harm the health. In addition, in conjunction with various optimization solutions, such as using the bristle base made of a light-transmittance material to refract and guide light to adjust an illumination region, using bristles made of light guide fibers to perform low-loss and high-efficiency light guidance, and covering a light reflection layer at an inner surface of the toothbrush head accommodating chamber to enhance the illumination intensity, the oral phototherapy effect is greatly enhanced, and a need of a user for deep oral care.

### BRIEF DESCRIPTION OF THE DRAWINGS

FIG. 1 is a structural exploded view of the present disclosure; and

FIG. 2 is a schematic structural diagram of the present disclosure.

In the drawings: 1: toothbrush head; 11: toothbrush head body; 12: toothbrush head housing; 13: toothbrush head accommodating chamber; 14: light reflection layer; 15: light emission lamp panel; 151: phototherapy LED; 16: bristle base; 17: bristle; 18: power receiving coil; 19: connecting end piece; 2: toothbrush handle; 21: handle housing; 22: power transmitting coil; 23: waterproof cover; 24: upper end plug; 25: driving motor; 26: storage battery; 27: bottom end plug; 28: electrical appliance support; 281: motor support; 282: battery support; 29: circuit board assembly; 291: control button; and 292: indicator light.

### DETAILED DESCRIPTION OF THE EMBODIMENTS

The technical solutions in the embodiments of the present disclosure will be clearly and completely described below with reference to the drawings in the embodiments of the present disclosure, and it is obvious that the described embodiments are only a part of the embodiments of the present disclosure but not all of them. Based on the embodiments of the present disclosure, all other embodiments obtained by those of ordinary skilled in the art without creative efforts fall within the protection scope of the present disclosure.

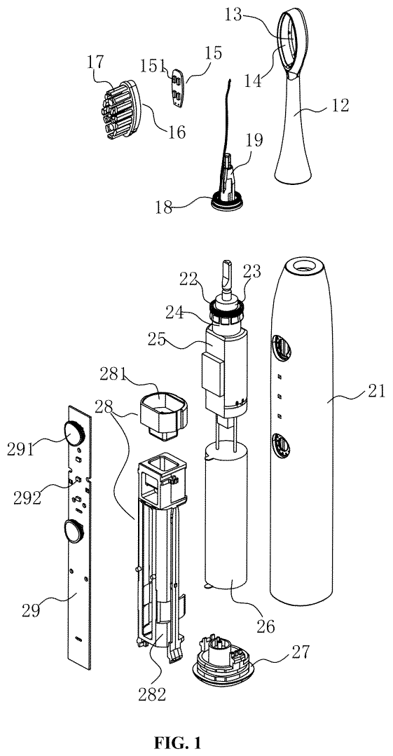

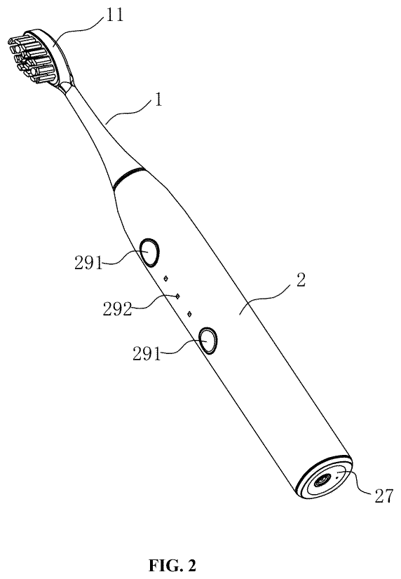

Referring to FIG. 1 to FIG. 2, a phototherapy care toothbrush includes a toothbrush head 1 and a toothbrush handle 2. The toothbrush head 1 is detachably connected to the toothbrush handle 2. A toothbrush head body 11 is arranged at one end of the toothbrush head 1, and a connecting end piece 19 is arranged at the other end. The toothbrush head 1 is detachably connected to an output shaft of a driving motor 25 on the toothbrush handle 2 through the connecting end piece 19. The toothbrush head body 11 includes a toothbrush head accommodating chamber 13 and a bristle base 16 covered at an opening of the toothbrush head accommodating chamber 13. The bristle base 16 is encapsulated at an opening of the toothbrush head accommodating chamber 13 by an ultrasonic process, and the bristle base 16 is connected to the toothbrush head accommodating chamber 13 in a water sealing manner. Bristles 17 are arranged on one side of the bristle base 16 away from the toothbrush head accommodating chamber 13. Ends of the bristles 17 away from the toothbrush head accommodating chamber 13 extend out and are placed outside the bristle base 16, and ends facing the toothbrush head accommodating chamber 13 penetrate through the bristle base 16 and are communicated to the toothbrush head accommodating chamber 13. A light emission lamp panel 15 is arranged in the toothbrush head accommodating chamber 13. The light emission lamp panel 15 is encapsulated with phototherapy LEDs 151. The phototherapy LEDs 151 include blue-light LEDs and red-light LEDs; a light wavelength of the blue-light LEDs is 400 to 460 nm; and a light wavelength of the red-light LED is 630 to 660 nm. Blue light in this wave band can effectively whiten the teeth, and red light in this wave band can effectively perform anti-inflammatory oral care. A direction of light emission of the phototherapy LEDs 151 is towards one side of the bristle base 16. An inner surface of the toothbrush head accommodating chamber 13 is covered with a light reflection layer 14. The light reflection layer 14 can reflect light of a diffuse reflection part of the phototherapy LEDs 151 towards the bristle base 16 to enhance the light intensity. The bristle base 16 is a light-transmittance member. A refractive index of the bristle base 16 can be adjusted according to an actual illumination range during the design of the product, so as to enlarge or narrow an illuminated region of phototherapy ray. The bristles 17 are light guide fibers. The light guide fibers can efficiently and accurately guide the ray to the teeth, which is beneficial to enhance the phototherapy care effect and reduce propagation loss of the ray. The toothbrush head 1 and toothbrush handle 2 are electrically connected to each other through an electrical connection mechanism. The electrical connection mechanism includes a power receiving coil 18 arranged in the toothbrush head 1 and a power transmitting coil 22 arranged on the toothbrush handle 2. The power receiving coil 18 and the power transmitting coil 22 are wirelessly electrically connected to each other.

The toothbrush head 1 includes a toothbrush head housing 12. The toothbrush head accommodating chamber 13 is formed in one end of the toothbrush head housing 12, and a conical cavity is formed in the other end. The connecting end piece 19 is embedded in the conical cavity. The power receiving coil 18 is arranged on one side of the connecting end piece 19 facing the conical cavity. The toothbrush handle 2 includes a handle housing 21. The handle housing 21 is a sleeve with openings provided in two ends. An upper end plug 24 is arranged at the opening in the upper end of the handle housing 21. The driving motor 25 is mounted in the handle housing 21. An output shaft of the driving motor 25 penetrates through the upper end plug 24 and extends outwards from the opening in the upper end of the handle housing. The upper end plug 24 is provided with the power transmitting coil 22, and the power receiving coil 18 is wirelessly electrically connected to the power transmitting coil 22.

An electrical appliance support 28 is arranged in the handle housing 21. The electrical appliance support 28 includes a motor support 281 and a battery support 282. An end portion of the motor support 281 and an end portion of the battery support 282 opposite to the end portion of the motor support 281 are connected in an inserted manner. A circuit board assembly 29 and a storage battery 26 are arranged in the handle housing 21. The driving motor 25, the storage battery 26, and the circuit board assembly 29 are fixedly mounted on the electrical appliance support 28. A waterproof cover 23 is encapsulated at the opening in the upper end of the handle housing 21. The waterproof cover 23 sleeves the output shaft of the driving motor in a circumferentially direction and is clamped between the upper end plug 24 and the handle housing 21. A bottom end plug 27 is encapsulated at the opening in the lower end of the handle housing 21. A power input port is arranged on the bottom end plug 27. The power input port is electrically connected to the circuit board assembly 29. The circuit board assembly 29 is electrically connected with control buttons 291 and indicator lights 292. The control buttons 291 and the indicator lights 292 are arranged in preset through holes of the handle housing 21. The control buttons 291 are configured to adjust a working mode of the driving motor 25 and a working mode of the phototherapy LEDs 151. The indicator lights 292 are configured to display a real-time working mode of the electric toothbrush.

In summary, according to the present disclosure, the phototherapy LEDs 151 are arranged in a toothbrush head body 11. The phototherapy LEDs 151 are used to emit phototherapy ray with a specific wavelength to assist in oral cleaning and oral care, so that a deep cleaning and care effect can be achieved. Furthermore, redundant chemical medicines can be effectively prevented from entering an internal system of a person from the mouth to harm the health. In addition, in conjunction with various optimization solutions, such as using the bristle base 16 made of a light-transmittance material to refract and guide light to adjust an illumination region, using the bristles 17 made of light guide fibers to perform low-loss and high-efficiency light guidance, and covering the light reflection layer 14 at the inner surface of the toothbrush head accommodating chamber 13 to enhance the illumination intensity, the oral phototherapy effect is greatly enhanced, and a need of a user for deep oral care.

It should be noted that in this document, relationship terms such as first and second are used solely to distinguish one entity or operation from another entity or operation without necessarily requiring or implying any actual such relationship or order between such entities or operations. Furthermore, the terms “include”, “including”, or any other variation thereof, are intended to encompass a non-exclusive inclusion, such that a process, method, article, or device that includes a list of elements does not include only those elements but may include other elements not explicitly listed or inherent to such process, method, article, or device.

Although the embodiments of the present disclosure have been shown and described, it can be understood by those of ordinary skill in the art that various changes, modifications, substitutions, and variations can be made to these embodiments without departing from the principle and spirit of the present disclosure. The scope of the present disclosure is defined by the accompanying claims and their equivalents.

What is claimed is:

1. A phototherapy care toothbrush, comprising a toothbrush head (1) and a toothbrush handle (2), wherein the toothbrush head (1) is detachably connected to the toothbrush handle (2); a toothbrush head body (11) is arranged at one end of the toothbrush head (1), and a connecting end piece (19) is arranged at the other end; the toothbrush head (1) is detachably connected to an output shaft of a driving motor (25) on the toothbrush handle (2) through the connecting end piece (19); the toothbrush head body (11) comprises a toothbrush head accommodating chamber (13) and a bristle base (16) covered at an opening of the toothbrush head accommodating chamber (13); bristles (17) are arranged on one side of the bristle base (16) away from the toothbrush head accommodating chamber (13); a light emission lamp panel (15) is arranged in the toothbrush head accommodating chamber (13); the light emission lamp panel (15) is encapsulated with phototherapy LEDs (151); a direction of light emission of the phototherapy LEDs (151) is towards one side of the bristle base (16); the bristle base (16) is a light-transmittance member, and the bristles (17) are light guide fibers; and the toothbrush head (1) and toothbrush handle (2) are electrically connected to each other through an electrical connection mechanism.

2. The phototherapy care toothbrush according to claim 1, wherein the bristle base (16) is sealed at the opening of the toothbrush head accommodating chamber (13) by water.

3. The phototherapy care toothbrush according to claim 1, wherein ends of the bristles (17) away from the toothbrush head accommodating chamber (13) extend out and are placed outside the bristle base (16), and ends facing the toothbrush head accommodating chamber (13) penetrate through the bristle base (16) and are communicated to the toothbrush head accommodating chamber (13).

4. The phototherapy care toothbrush according to claim 1, wherein an inner surface of the toothbrush head accommodating chamber (13) is covered with a light reflection layer (14).

5. The phototherapy care toothbrush according to claim 1, wherein the phototherapy LEDs (151) comprise blue-light LEDs and red-light LEDs; a light wavelength of the blue-light LEDs is 400 to 460 nm; and a light wavelength of the red-light LED is 630 to 660 nm.

6. The phototherapy care toothbrush according to claim 1, wherein the electrical connection mechanism comprises a power receiving coil (18) arranged in the toothbrush head (1) and a power transmitting coil (22) arranged on the toothbrush handle (2); and the power receiving coil (18) and the power transmitting coil (22) are wirelessly electrically connected to each other.

7. The phototherapy care toothbrush according to claim 6, wherein the toothbrush head (1) comprises a toothbrush head housing (12); the toothbrush head accommodating chamber (13) is formed in one end of the toothbrush head housing (12), and a conical cavity is formed in the other end; the connecting end piece (19) is embedded in the conical cavity; the power receiving coil (18) is arranged on one side of the connecting end piece (19) facing the conical cavity; the toothbrush handle (2) comprises a handle housing (21); the handle housing (21) is a sleeve with openings provided in two ends; an upper end plug (24) is arranged at the opening in the upper end of the handle housing (21); the driving motor (25) is mounted in the handle housing (21); an output shaft of the driving motor (25) penetrates through the upper end plug (24) and extends outwards from the opening in the upper end of the handle housing; the upper end plug (24) is provided with the power transmitting coil (22); and the power receiving coil (18) is wirelessly electrically connected to the power transmitting coil (22).

8. The phototherapy care toothbrush according to claim 7, wherein an electrical appliance support (28) is arranged in the handle housing (21); a circuit board assembly (29) and a storage battery (26) are arranged in the handle housing (21); the driving motor (25), the storage battery (26), and the circuit board assembly (29) are fixedly mounted on the electrical appliance support (28); a waterproof cover (23) is encapsulated at the opening in the upper end of the handle housing (21); the waterproof cover (23) sleeves the output shaft of the driving motor in a circumferentially direction and is clamped between the upper end plug (24) and the handle housing (21); a bottom end plug (27) is encapsulated at the opening in the lower end of the handle housing (21); a power input port is arranged on the bottom end plug (27); and the power input port is electrically connected to the circuit board assembly (29).

9. The phototherapy care toothbrush according to claim 8, wherein the circuit board assembly (29) is electrically connected with control buttons (291) and indicator lights (292); the control buttons (291) and the indicator lights (292) are arranged in preset through holes of the handle housing (21).

10. The phototherapy care toothbrush according to claim 8, wherein the electrical appliance support (28) comprises a motor support (281) and a battery support (282); and an end portion of the motor support (281) and an end portion of the battery support (282) opposite to the end portion of the motor support (281) are connected in an inserted manner.
